(19) United States
(12) Patent Application Publication
(10) Pub. No.: US 2025/0171763 A1
(43) Pub. Date: May 29, 2025

(54) METHOD FOR PURIFYING A PATHOGEN GENOME SAMPLE

(71) Applicant: Research Foundation of the City University of New York, New York, NY (US)

(72) Inventor: Sherin Kannoly, Kew Gardens, NY (US)

(21) Appl. No.: 18/877,713

(22) PCT Filed: Jun. 28, 2023
(86) PCT No.: PCT/US23/26429

§ 371 Date: Dec. 20, 2024

Related U.S. Application Data
(60) Provisional application No. 63/356,098, filed on Jun. 28, 2022.

(51) Int. Cl. C12N 15/10

(52) U.S. Cl. CPC C12N 15/1003

(57) ABSTRACT

A method for concentrating a liquid sample containing pathogens. The method traps pathogens on the surface of activated carbon. Prior to release of the pathogens, the activated carbon is treated with a blocking solution that prevents the activated carbon from degrading and/or binding the released pathogen nucleic acids. After treatment with the blocking solution, the pathogens are released from the activated carbon with a lysis reagent.

### CROSS-REFERENCE TO RELATED APPLICATIONS

This application claims priority to, and is a non-provisional of, U.S. Patent Application 63/356,098 (filed Jun. 28, 2022), the entirety of which is incorporated herein by reference.

### STATEMENT OF FEDERALLY SPONSORED RESEARCH OR DEVELOPMENT

This invention was made with government support under grant number UD-48215 awarded by the National Institute of Health. The government has certain rights in the invention.

### BACKGROUND OF THE INVENTION

Activated carbon has been long known to adsorb viruses, which may retain their infectivity even after adsorption. However, upon release from the carbon, the viral genomes appear to be either adsorbed and/or destroyed by the activated carbon. The poor performance of this conventional methodology is demonstrated in an experiment where a conventional method of viral concentration using polyethylene glycol (PEG) was compared to activated carbon (FIG. 1).

A sample of bovine corona virus was exposed to activated carbon. The TRIzol Reagent (INVITROGEN®) was used for releasing viral RNA. However, no genetic signal of bovine coronavirus (BCoV) suspended in phosphate buffered saline (PBS) (MP Biomedicals) was detected using the activated carbon method. Thus, the conventional carbon-based method is clearly unsuitable for genome extractions.

FIG. 1 also presents a conventional PEG method for concentrating viruses. By omitting activated carbon, significant amounts of viral RNA could be isolated. While the PEG method is suitable in some situations, an improved method is therefore desired that provides a wider range of options for concentrating pathogen genomes.

The discussion above is merely provided for general background information and is not intended to be used as an aid in determining the scope of the claimed subject matter.

### SUMMARY

This disclosure provides a method for purifying a pathogen genome sample. The method traps pathogen particles on the surface of activated carbon. Prior to release of the pathogen particles, the activated carbon is treated with a blocking solution that prevents the activated carbon from degrading and/or binding the released pathogen nucleic acids. After treatment with the blocking solution, the pathogen particles are released from the activated carbon with a lysis reagent.

The technical problem to be solved is the inability of conventional activated carbon methods to extract pathogen genomes without degradation and/or loss due to binding to the carbon surface. An advantage that may be realized in the practice of some embodiments of the method is that the method permits the use of activated carbon to extract pathogen genomes with dramatically reduced viral genome loss due to degradation and/or binding to the carbon surface, relative to conventional carbon-based attempts. Another advantage that may be realized in the practice of some embodiments of the method is that the method provides higher degrees of concentration than conventional PEG methods.

In a first embodiment, a method for purifying a pathogen genome sample is provided. The method comprises sequential steps of: a) exposing activated carbon to a sample comprising a pathogen, thereby forming a carbon-pathogen complex; b) treating the carbon-pathogen complex with a blocking solution comprising a buffered, aqueous solution of amino acids; c) waiting a predetermined amount of time; d) removing the blocking solution from the carbon-pathogen complex; e) washing the carbon-pathogen complex with a buffered solution, the step of washing occurring at least once; f) treating with a lysis reagent to produce a released pathogen genome sample; and g) purifying the released pathogen genome sample.

This brief description of the invention is intended only to provide a brief overview of subject matter disclosed herein according to one or more illustrative embodiments, and does not serve as a guide to interpreting the claims or to define or limit the scope of the invention, which is defined only by the appended claims. This brief description is provided to introduce an illustrative selection of concepts in a simplified form that are further described below in the detailed description. This brief description is not intended to identify key features or essential features of the claimed subject matter, nor is it intended to be used as an aid in determining the scope of the claimed subject matter. The claimed subject matter is not limited to implementations that solve any or all disadvantages noted in the background.

### BRIEF DESCRIPTION OF THE DRAWINGS

So that the manner in which the features of the invention can be understood, a detailed description of the invention may be had by reference to certain embodiments, some of which are illustrated in the accompanying drawings. It is to be noted, however, that the drawings illustrate only certain embodiments of this invention and are therefore not to be considered limiting of its scope, for the scope of the invention encompasses other equally effective embodiments. The drawings are not necessarily to scale, emphasis generally being placed upon illustrating the features of certain embodiments of the invention. In the drawings, like numerals are used to indicate like parts throughout the various views. Thus, for further understanding of the invention, reference can be made to the following detailed description, read in connection with the drawings in which:

FIG. 1 is a graph showing detection of bovine coronavirus reconstituted in PBS buffer and concentrated using activated carbon and a conventional method that uses polyethylene glycol (PEG).

FIG. 2A depicts one possible mechanism of nucleic acids released from virus particles adsorbed onto solid phase exchange materials (SPEM) such as activated carbon.

FIG. 2B is a graph comparing bovine coronavirus (BCoV) titers extracted using activated carbon, a conventional method that used polyethylene glycol (PEG) and the disclosed method.

FIG. 3 is a schematic of one filter for use with the disclosed method.

FIG. 4 is a graph comparing different methods to quantify SARS-CoV-2 RNA from wastewater samples obtained from two wastewater treatment plants (labeled 1 and 2) from New York City (NYC). The pair of black bars in the middle are samples collected on a different day.

FIG. 5 is a graph showing stability of SARS-CoV-2 isolated from wastewater 48 h after adsorption. The carbon-adsorbed viruses were incubated for 48 h in blocking solution before lysis and RNA extraction.

FIG. 6 is a graph showing recovery of Influenza A virus from a wastewater treatment plant in New York City (NYC) using the disclosed method.

FIG. 7 is a graph showing detection of Pepper mild mottle virus (PMMoV) from wastewater samples obtained from two wastewater treatment plants in New York City (NYC).

FIG. 8 is a graph assessing infectivity of lambda phage after adsorption onto carbon particles.

FIG. 9A and FIG. 9B are graphs showing detection of viruses from samples concentrated using an in situ trap.

FIG. 10 is a graph showing detection of SARS-CoV-2 in hospital wastewater using an in situ trap.

FIG. 11 is a graph showing detection of MPX in hospital wastewater using an in situ trap.

FIG. 12 is a graph showing detection of Influenza A in hospital wastewater using an in situ trap.

FIG. 13 is a schematic depiction of one pathogen trap for capturing viruses from water.

FIG. 14 is a schematic depiction of one pathogen trap for capturing aerosolized viruses.

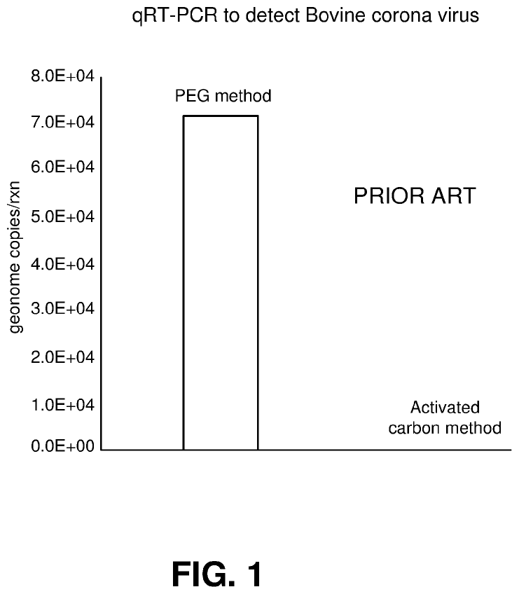

### DETAILED DESCRIPTION OF THE INVENTION

This disclosure provides a method for purifying and protecting pathogen particles from a large volume and, in some embodiments, the subsequent extraction of pathogen genomes. The method uses a quick solid-phase exchange of pathogens suspended in solutions of low titer such as wastewater and environmental samples.

The method is initiated by exposing activated carbon to a sample comprising a pathogen to form a carbon-pathogen complex. Examples of suitable activated carbons include activated charcoal (e.g. Sigma-Aldrich, C4386), which is granular carbon with high porosity, for adsorbing viruses. The majority of carbon particles (more than 70-75%) are greater than 10 microns in diameter and more than 10-15% are greater than 74 microns in diameter with a surface area of approximately 1000-1200 (e.g. about 1100) m² per g. In one embodiment, the exposing continues for at least 15 minutes. In another embodiment, the exposing continues at least 30 minutes. Activated fine carbon has different kinds of pores namely micropores (<2 nm in diameter), mesopores (2-50 nm) and macropores (>50 nm). The percentage of these pores, and therefore the surface area available for binding, vary depending on the type of carbon and could be used to adsorb different molecules other than viruses.

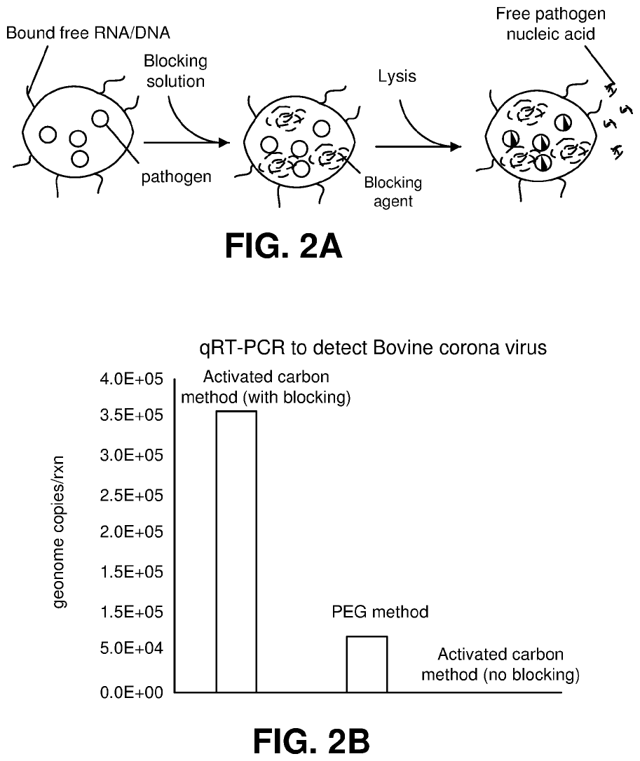

After the carbon-pathogen complex is formed, the complex is treated with a blocking solution comprising buffered, aqueous amino acids (including peptides). In one embodiment, the amino acids are present in the blocking solution at a concentration of at least 1% (m/v). In another embodiment, the amino acids are present in the blocking solution at a concentration of at least 5% (m/v). In another embodiment, the amino acids are present in the blocking solution at a concentration of at least 10% (m/v). In yet another embodiment, the amino acids are present in the blocking solution at a concentration of at least 20% (m/v). Referring to FIG. 2A, without wishing to be bound to any particular theory, the amino acids are believed to block potential nucleic acid-binding sites on the active carbon. This blocking is achieved by facilitating the binding of amino acids and polypeptides to the potential nucleic acid-binding sites. For example, a 20% (m/v) solution of tryptone (e.g. BACTO® Tryptone, GIBCO®) in PBS may be used as the blocking solution (BS). Blocking solutions that block specific adsorption sites can be designed to facilitate selective adsorption of molecules of interest. Such targeted designs could be employed as molecular sieves to separate and/or purify molecules of interest. For blocking solutions, alternatives to tryptone including but not limited to other partially or fully digested protein products such as peptides, protein hydrolysates, peptone, casamino acids, yeast extract, beef extract, gelatin, meat extract, casein hydrolysate, corn steep liquor, hydrolyzed vegetable proteins (soy, wheat, or corn), soy peptone, atholate (plant protein hydrolysate) etc. can be used. Also, for blocking larger sites, proteins including but not limited to lactalbumin, bovine serum albumin, egg albumin, whey protein, casein protein, soy protein, pea protein, rice protein, hemp protein, pumpkin seed protein, sunflower seed protein, peanut protein, sesame seed protein, almond protein etc. may be employed.

After the carbon-pathogen complex is treated with the blocking solution, the user waits for a predetermined amount of time (e.g. at least 1 minute) to permit absorption of the amino acid. In one embodiment, the predetermined amount of time is at least 5 minutes. In another embodiment, the predetermined amount of time is at least 7 minutes.

The blocking solution is then removed by, for example, filtration such that the now blocked carbon-pathogen complex is separated from the blocking solution. The steps of treating with the blocking solution, waiting a predetermined amount of time, and then removing the blocking solution are repeated at least once (i.e. at least two occurrences of the steps). In one embodiment, these steps are repeated at least twice (i.e. at least three occurrences of the steps). In yet another embodiment, these steps are repeated at least three times (i.e., at least four occurrences of the steps).

The carbon-virus complex, after having been removed from the blocking solution, is then washed with a buffer solution at least once. In another embodiment, the washing occurs at least twice. In another embodiment, the washing occurs at least three times. In one embodiment, the buffer solution is a phosphate buffered saline (PBS) solution. Alternatively, other buffers such as Tris-buffered saline, HEPES-buffered saline (N-(2-hydroxyethyl)piperazine-N′-(2-ethanesulfonic acid)), MOPS-buffered saline (3-morpholinopropane-1-sulfonic acid), MES (2-(N-morpholino)ethanesulfonic acid), tricine-buffered saline, etc., which stabilize nucleic acids, proteins, and cell membranes, may be used.

After buffering, the carbon-pathogen complex is then treated with a lysis reagent to release the encapsulated pathogen genome from the activated carbon. Examples of suitable lysis reagents include TRIzol Reagent (or lysis buffer if using commercially available kits). Examples of lysis reagents from kits include Buffer AVL (Qiagen), protease solution (Promega WIZARD® Enviro Total Nucleic Acid Kit), Viral DNA/RNA Buffer (Zymo research), etc.

The released pathogen genome is then purified by, for example, ethanol precipitation, column purification, or other suitable techniques.

Referring to FIG. 2B, the use of activated carbon without blocking showed no recovered viral genome. The use of conventional PEG methodology showed approximately 6.66×10⁴ genome copies recovered. In contrast, the use of the disclosed method showed 3.045×10⁵ genome copies recovered which is an improvement of almost an order of magnitude.

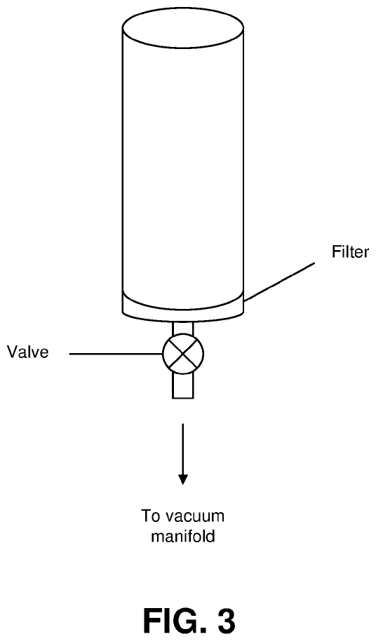

Experiments to generate the data of FIG. 2B were performed in flasks, where the reconstituted viral suspensions (100 ml) in PBS were agitated with 0.2 grams of activated carbon (Sigma-Aldrich, C4386) for half an hour. The majority of carbon particles used (70-75%) was greater than 10 microns in size and 10-15% was greater than 74 microns with a surface area of approximately 1100 square meters per gram. After adsorption, the contents were centrifuged (8000×g for 15 mins) to collect the carbon-virus complex. These centrifugation and resuspension steps were time consuming and labor intensive especially if a large number of samples needs to be processed. To mitigate these issues, a vacuum filter device was designed to trap the activated carbon in a filter paper (ThermoScientific, 88600) placed inside a column (FIG. 3).

### Example

Using a rotator drum, an ice-cold virus solution (40 ml) was first mixed with about 0.2 g of activated carbon for 30 min. The solution was then passed through the filter columns (FIG. 3) using a vacuum manifold, which was connected to a vacuum line. As the vacuum was applied, the solution passed through the filter thereby trapping the virus-adsorbed carbon particles.

A 1.5 ml aliquot of ice-cold blocking solution (20% tryptone in PBS) was added to the filter and incubated for 5 min. The blocking solution was removed by applying vacuum and a fresh aliquot of blocking solution was added and incubated for another 7.5 min. This blocking step was repeated four times, thus passing a total of 6 ml of BS through the filter-trapped activated carbon. The blocking was followed by two steps of washing with 1.5 ml of PBS.

The TRIzol reagent (0.75 ml twice) was then added as a lysis reagent to the filter to open viral capsids and release the genomes. Vacuum was applied to collect TRIzol with viral genomes, which were then precipitated as per the manufacturer's instructions.

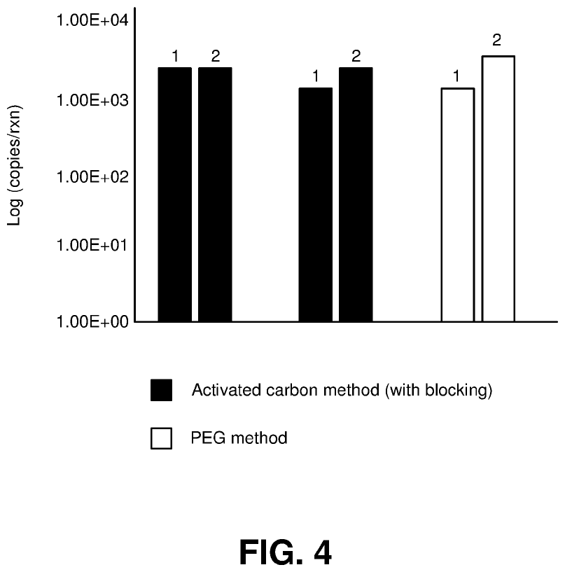

Referring to FIG. 4, the disclosed method was used to concentrate SARS-CoV-2 and purify viral RNA from New York city (NYC) wastewater samples. The samples were collected by the Department of Environmental Protection (DEP) from various wastewater treatment plants (WWTP) as part of their surveillance program.

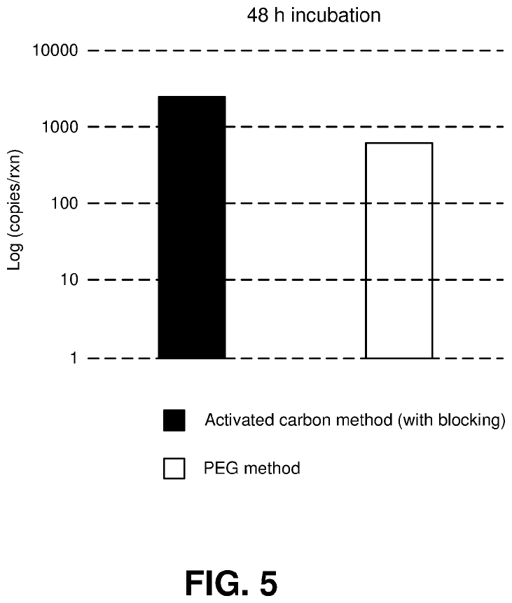

Referring to FIG. 5, the carbon-adsorbed SARS-CoV-2 viruses can be stored in blocking solution (20% tryptone in PBS) for a period of at least 48 h at 4° C. without any loss of genetic signal. The adsorbed viruses are stable in the blocking solution for 48 h at 4° C. This is noteworthy because the virus appears to rapidly degrade overnight in pasteurized wastewater stored at 4° C. (data not shown).

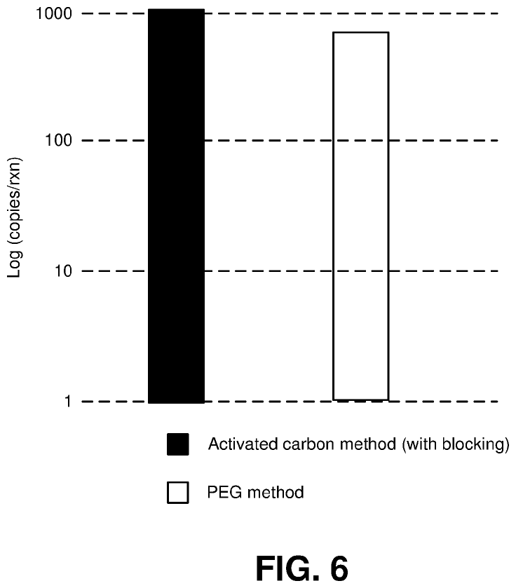

Referring to FIG. 6, the method was also used to concentrate Influenza A virus and purify viral RNA from a wastewater treatment plant in New York City (NYC) showing results comparable to the PEG method.

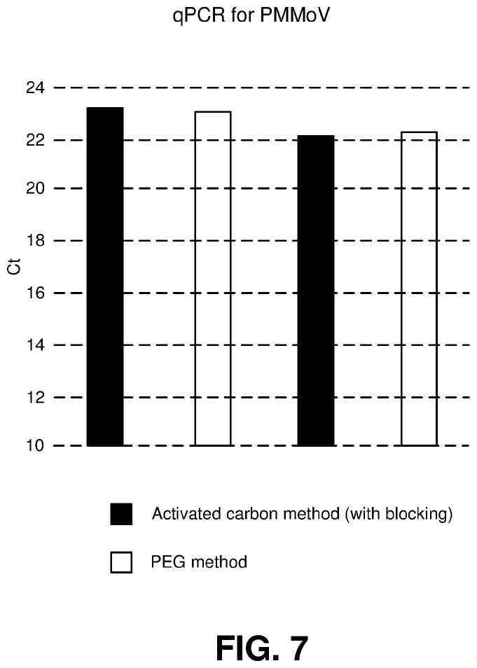

Additionally, the disclosed method was successfully used to concentrate the plant virus Pepper mild mottle virus (PMMoV) and purify viral RNA, which is an indicator of fecal contamination and is routinely used as an internal control in wastewater analysis (FIG. 7).

### Infectivity Assay

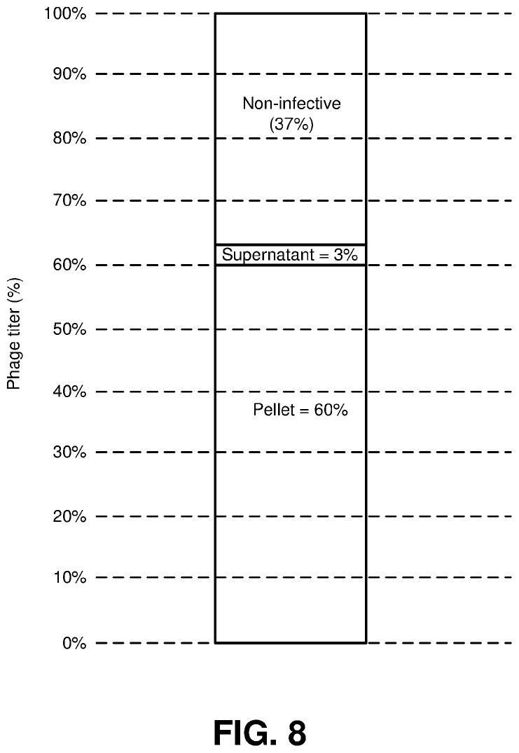

An infectivity assay was performed using lambda, a nonenveloped bacteriophage (phage). The lambda phage was adsorbed onto activated carbon and the infectivity was measured using plaque assays. The supernatant obtained after pelleting the carbon was also tested for infectivity using plaque assays to determine the proportion of phages that was adsorbed to activated carbon particles. After resuspension and plaque assays, 60% of lambda phage was detected in the activated carbon pellet (FIG. 8) and only 3% in the supernatant.

### Pathogen Trap

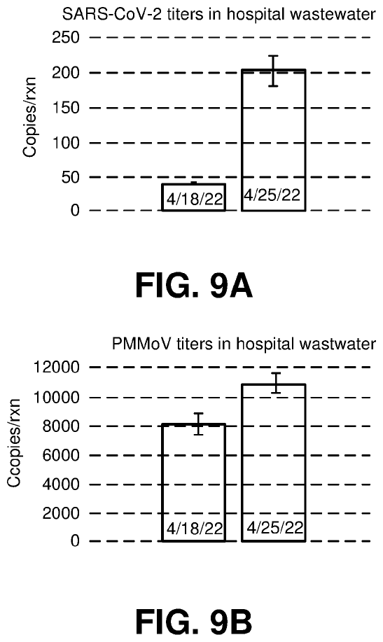

An in situ pathogen trap was designed by immobilizing activated carbon particles into housings (e.g. steel housings). These traps can be deployed into wastewater sources such as sewer outflows or manholes. In one set of experiments, in situ virus traps were used in wastewater outflows of New York City (NYC) hospitals. After placing the traps for 10 h in manholes, SARS-CoV-2 viruses were successfully concentrated, which was followed by purification and quantification of the viral RNA genomes (FIG. 9A and FIG. 9B). PMMoV viruses were also detected from the same traps indicating the ability of trapping different viruses at the same time. Advantageously, the in situ virus trap concentrates viruses at the source without the need for transporting hazardous wastewater into labs for processing. The trap is designed to replace composite autosamplers, which use large pumps installed near manholes to collect aliquots of wastewater over a long period of time. In contrast to the autosamplers, the in situ virus trap is much smaller and can be easily deployed inside a manhole. RNA isolated from in situ traps installed inside manholes were successfully sequenced to identify variants.

By way of illustration, 0.8 g of 0.8 mm pellets of activated carbon (Norit ROW, fisher scientific, cat no. AAL1633422) was packed and heat scaled into a perforated nylon sachet. The sealed sachets were placed into perforated steel housings. The housing produced the sachet and the activated carbon within while the perforations permitted exposure to the environment. Several assembled housings were held together in a bunch using drilled holes and nylon rope.

The assembled housings, after being tied in a bunch, can be easily deployed into water sources such as sewage manholes or natural water bodies such as lakes or rivers. This design minimizes resistance to flowing water and the cartridges are free to move independently of each other. This prevents the accumulation of solid debris over the cartridges and allows efficient capture of target microorganisms.

The assembled housings were deployed into sewage manholes or main pipes in hospitals across New York City. The devices were left in wastewater for 24 h and, thereafter, the nylon ropes were cut to transfer the housings into ice cold phosphate buffered saline (PBS). To isolate the activated carbon and any microorganisms thereon, a filter column (whatman filter paper fixed to the bottom of a 50 ml syringe) containing 50 ml PBS attached to a vacuum manifold was used. The sachet was removed from the housing and rinsed in PBS to remove any attached solids. The sachet was then cut open and gently shaken in the filter column containing PBS to resuspend the activated carbon. Then vacuum is applied to allow all the PBS to drain leaving the activated carbon trapped in the filter.

The activated carbon was then soaked in 5 ml blocking solution (20% tryptone in PBS) for 5 min. Vacuum was applied to drain the blocking solution and this was repeated for a total of four soakings. After draining the blocking solution, 5 ml of PBS was added to the carbon particles to soak for 2 min. Vacuum was applied to drain the PBS and a fresh 5 ml aliquot is added. This was repeated once more, and the vacuum was applied for an extra 30 s to remove any extra PBS. Several 10-ml aliquots of deionized (DI) water were used to transfer all the particles from the filter column into a fresh 50 ml tube. The tube was decanted to remove all DT water and 10 ml of fresh DT water was added to the carbon particles for total nucleic acid extraction using the Promega WIZARD® Enviro TNA kit. 200 μl of protease provided in the kit was then added followed by gentle swirling and incubation at room temperature for 30 min. 3 ml of binding buffer 1 (BBD), 250 μl of Binding buffer 2 (BBE), and 12 ml of isopropanol was added, and the contents were mixed by gently inverting the tubes. The tubes were then centrifuged at 3000×g for 10 mins. The supernatant was decanted into a Promega PUREYIELD™ binding column taking care to not transfer any activated carbon. Vacuum was then applied until all the liquid is drained. Subsequently, the steps in the kit manual were followed to extract total nucleic acids in an elution volume of 80 μl.

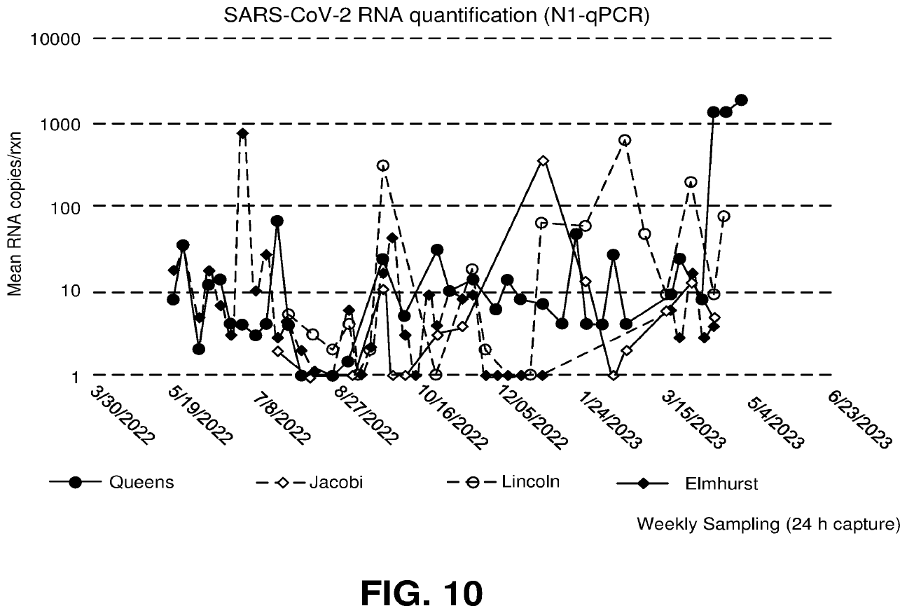

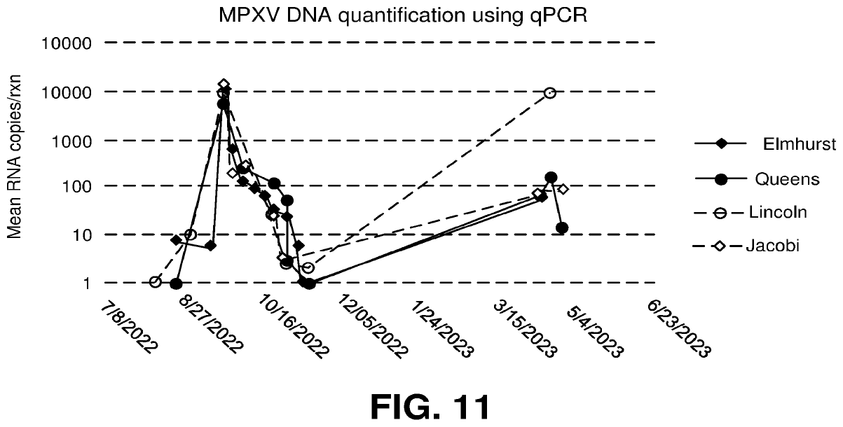

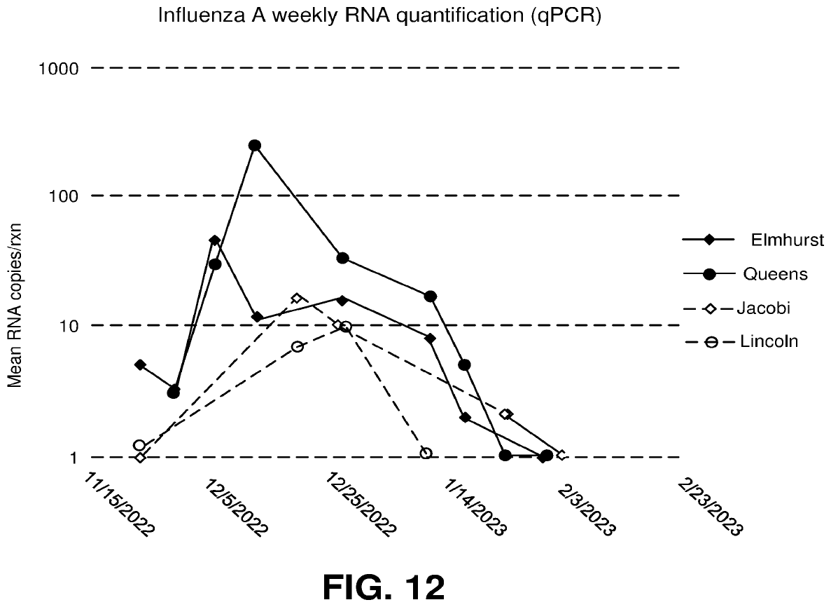

These devices were deployed once every week in manholes or main pipes from where the hospital wastewater flowed into the city sewers. The devices deployed at four hospitals continuously captured targets for a period of 24 h. Quantitative RT-PCR was used to detect the presence of specific targets: SARS-CoV-2 (FIG. 10), MPX (FIG. 11), and influenza (FIG. 12) for several months. The total nucleic acids were also used for shotgun sequencing, which enabled the detection of a wide range of bacterial and viral species including clinically relevant pathogens (see Table 1 for summary of relevant pathogens). Two samples collected from Elmhurst seven days apart were sequenced, which generated 70 and 50 million read pairs for the respective samples. Kraken2 analyses using the mini database (8 Gb) were able to classify about 50% of the reads with majority from bacteria (99%) and a small portion from viruses (less than 1%).

TABLE 1
Metagenomic sequencing of wastewater
nucleic acid obtained from Elmhurst

| Pathogen | # Reads | Comments |
|---|---|---|
| Acinetobacter spp. | 1568750 | |
| Acinetobacter | 20051 | ESKAPE pathogen |
| baumannii | | |
| Pseudomonas | 13210 | ESKAPE pathogen |
| aeruginosa | | |
| Klebsiella | 61688 | ESKAPE pathogen |
| pneumoniae | | |
| Enterobacter spp. | 70073 | ESKAPE pathogen |
| Escherichia coli | 16687 | Various ETEC and EHEC strains e.g. |
| | | O157:H7, O104:H4 |
| Salmonella enterica | 5618 | |
| Yersinia | 1423 | Yersinia pseudotuberculosis and |
| | | pestis, “Plague” bacteria |
| Stenotrophomonas | 1620 | Emerging MDR associated with HAIs |
| maltophilia | | |
| Vibrio cholerae | 922 | O1 serogroup |
| Haemophilus | 600 | |
| parainfluenzae | | |
| Legionella | 1007 | Legionnaires' disease |
| Francisella tularensis | 367 | Tularemia |
| Bordetella | 126 | Whooping cough |
| pertussis/parapertussis | | |
| Shewanella | 6214 | increasing incidences of |
| | | Shewanella infection and |
| | | the emerging drug resistance |
| | | of Shewanella strains |
| Aggregatibacter | 215 | |
| Comamonas | 42418 | Emerging pathogen |
| Delftia tsuruhatensis | 760 | can cause infection in immunocompetent |
| | | and immunocompromised individuals |
| Cupriavidus | | |
| Burkholderia | 493 | Mclioidosis |
| pscudomallci | | |
| Pandoraea | 1739 | Most of Pandoraea species are |
| | | associated with lung |
| | | infections in cystic fibrosis patients |
| Achromobacter | 1433 | broad range of infections in hosts with |
| | | other underlying conditions |
| Herbaspirillum | 798 | Herbaspirillum species are capable |
| | | of causing bacteremia and sepsis in |
| | | immunocompromised patients |
| Neisseria meningitidis | 376 | |
| Campylobacter | 5776 | People with Campylobacter |
| | | infection usually have diarrhea (often |
| | | bloody), fever, and stomach cramps |
| Helicobacter | 518 | |
| Methylobacterium | | Most reported Methylobacterium |
| | | infections have been nosocomial. |
| Myroides | 3888 | opportunistic pathogens |
| Clostridioides | 484 | |
| difficile | | |
| Clostridium | 548 | |
| perfringens | | |
| Clostridium | 460 | |
| botulinum | | |
| Bacillus anthracis | 199 | |
| Staphylococcus | 636 | |
| aureus | | |
| Chlamydia | 27 | |
| trachomatis | | |
| Fusobacterium | 53 | rare however serious infections |
| | | with complications |
| necrophorum | | or mortality may occur occasionally |
| Fusobacterium | 51 | |
| nucleatum | | |
| Treponema pallidum | 1 | |
| Other signals | | |
| Homo sapiens | 485165 | |
| Archaea | 1107 | |
| Viruses | 3838 | |
| ds DNA viruses | 1578 | |
| ss RNA viruses | 117 | |
| ssDNA viruses | 34 | |

Advantageously, this in situ pathogen trap does not require power supply or pumps to draw wastewater and can be deployed by personnel who do not need extensive training or expertise thus making it user-friendly and affordable compared to active samplers. Furthermore, the carbon-based traps dispense with the need to transport large volumes of hazardous wastewater to laboratories (as is currently done) as only the microbe-bound activated carbon (which weigh less than a gram) need to be brought, handled, and processed with minimum risk of exposure at the lab. The trap can be easily deployed at wastewater treatment plants, manholes, building-level pipes, and even open sewers and latrines when more granular surveillance of outbreaks is required. These devices continuously collect and concentrate pathogens while deployed, thereby increasing pathogen recovery in highly dilute samples. The devices are easily retrieved and safely transported to labs for further downstream processing. The downstream processing of captured pathogens is simpler and can be potentially automated for high throughput.

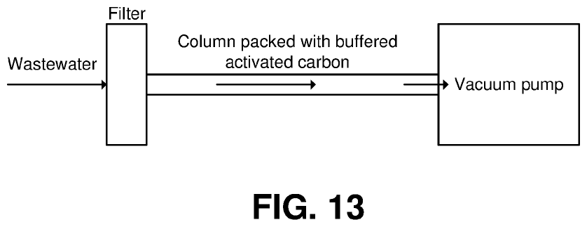

Referring to FIG. 13, a raw water sample may be automatically processed in a device such as the device of FIG. 13. The raw sample can be filtered, then passed through a column packed with activated carbon and subsequently this filtered water can be released back into the pipes. The viruses adsorbed inside the carbon column can be brought back for further processing, thus avoiding any liquid handling, splashing, or spillage.

In another embodiment, an automated or remote-controlled robot is used to collect wastewater samples from the pipes. Using such robots permits one to collect wastewater samples from locations (e.g. sewer pipes or manholes) closer to the source than a wastewater treatment plant and, thus, allows a more precise spatial resolution of disease outbreaks. Unlike the samples from a wastewater treatment plant, such samples may be relatively fresh and highly infectious raising safety concerns and requiring special handling and BSL3 labs for processing.

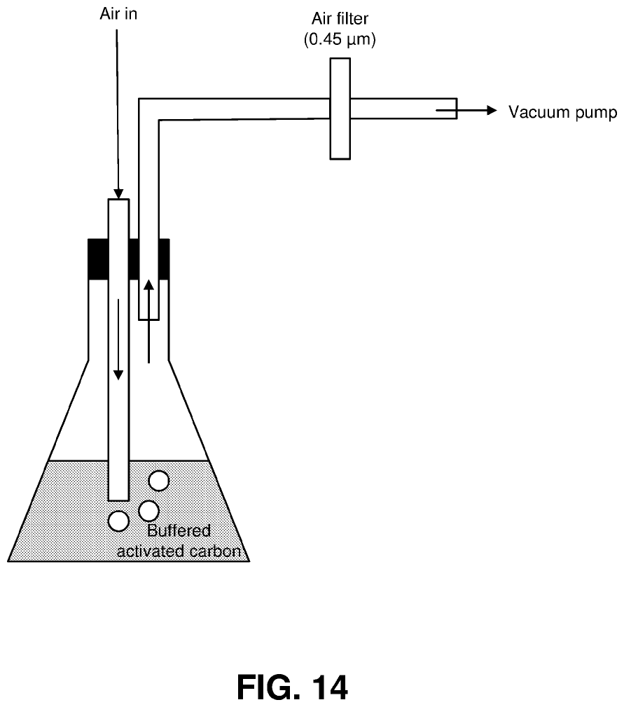

FIG. 14 depicts a trap designed for safely trapping viruses contained in aerosols. A vacuum pump draws air into an air trap containing activated carbon suspended in buffer. The air bubbles result in mixing of the activated carbon particles, maximizing the surface area available for adsorption. The aerosols entering the trap mix with the buffer allowing adsorption of viruses as well as other microorganisms on to the activated carbon particles. Although the air leaving the trap can also aerosolize, the high affinity of activated carbon minimizes the escape of adsorbed particles. The air leaving the trap can be passed through a filter to capture any aerosols that might possibly escape. Such traps could be used for surveillance of crowded indoor facilities as well as outdoors.

### Applications

Activated carbon can be used to adsorb a wide range of viruses from phages to SARS-CoV-2. Using the disclosed method, viruses from environmental samples can be easily concentrated for nucleic acid purification. This is a useful tool in virome studies of environments such as rivers, lakes, or oceans, where low viral titers require processing of large sample volumes. This method can also be used to concentrate other microorganisms, such as pathogenic bacteria, for subsequent RNA purification when they are present in extremely low numbers.

Several pathogenic viruses ranging from poliovirus to SARS are shed in human feces and can be tracked using samples from wastewater treatment plants. Continuous surveillance along with improved detection systems and sequencing capabilities can dramatically improve the preparedness for future epidemics or pandemics. Characterizing the viral genomes extracted from wastewater is an efficient method of tracking viral evolution. The major challenge in using this approach is obtaining high enough viral concentrations that allow high quality genome sequencing. The disclosed method allowed safe and efficient processing of large volumes of wastewater. Using the disclosed method in the lab, we were able to successfully concentrate viruses from volumes that were 6-25 fold greater than the volumes used in currently available methods. Unlike most methods that use volumes greater than the standard 40 ml, the disclosed method can be automated to process a large number of wastewater samples in a lab. Viral RNAs isolated using the disclosed method were successfully sequenced to identify variants.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A method for purifying a pathogen genome sample, the method comprising sequential steps of:
a) exposing an activated carbon to a sample comprising a pathogen, thereby forming a carbon-pathogen complex;
b) treating the carbon-pathogen complex with a blocking solution comprising a buffered, aqueous solution of amino acids;
c) waiting a predetermined amount of time;
d) removing the blocking solution from the carbon-pathogen complex;
e) washing the carbon-pathogen complex with a buffered solution, the step of washing occurring at least once;
f) treating with a lysis reagent to produce a released pathogen genome sample;
g) purifying the released pathogen genome sample.

2. The method as recited in claim 1, wherein the amino acids are present in the blocking solution at a concentration of at least 5% (m/v).

3. The method as recited in claim 1, wherein the amino acids are present in the blocking solution at a concentration of at least 20% (m/v).

4. The method as recited in claim 1, wherein the buffered, aqueous solution of amino acids comprises tryptone.

5. The method as recited in claim 1, wherein the buffered, aqueous solution of amino acids comprises a tryptone, a protein hydrolysate, a peptone, a casamino acid, a yeast extract, a beef extract, a gelatin, a meat extract, a casein hydrolysate, a corn steep liquor, a hydrolyzed vegetable protein, a soy peptone, an atholate, or combinations thereof.

6. The method as recited in claim 1, wherein the buffered, aqueous solution of amino acids comprises a lactalbumin, a bovine serum albumin, an egg albumin, a whey protein, a casein protein, a soy protein, a pea protein, a rice protein, a hemp protein, a pumpkin seed protein, a sunflower seed protein, a peanut protein, a sesame seed protein, an almond protein, or combinations thereof.

7. The method as recited in claim 1, wherein the activated carbon comprises pores wherein at least 70% are greater than 10 microns in diameter and at least 10% are greater than 74 microns in diameter.

8. The method as recited in claim 1, wherein the activated carbon provides a surface area of at least 1000 m² per g.

9. The method as recited in claim 1, wherein the activated carbon provides a surface area of between 1000 and 1200 m² per g.

10. The method as recited in claim 1, wherein the step of exposing the activated carbon to the sample persists for at least 15 minutes.

11. The method as recited in claim 1, wherein the step of exposing the activated carbon to the sample persists for at least 30 minutes.

12. The method as recited in claim 1, wherein the step of waiting the predetermined amount of time in step c) persists for at least 1 minute.

13. The method as recited in claim 1, wherein the step of waiting the predetermined amount of time in step c) persists for at least 5 minutes.

14. The method as recited in claim 1, wherein the step of waiting the predetermined amount of time in step c) persists for at least 7 minutes.

15. The method as recited in claim 1, further comprising repeating steps b) to d) at least once, the step of repeating occurring after step d).

16. The method as recited in claim 1, further comprising repeating steps b) to d) at least twice, the step of repeating occurring after step d).

17. The method as recited in claim 1, wherein the buffered solution comprises a phosphate buffered saline (PBS), a Tris-buffered saline, a N-(2-hydroxyethyl)piperazine-N′-(2-ethanesulfonic acid) (HEPES)-buffered saline, a 3-morpholinopropane-1-sulfonic acid (MOPS)-buffered saline, a 2-(N-morpholino)ethanesulfonic acid (MES), a tricine-buffered saline or combinations thereof.

18. The method as recited in claim 1, wherein the sample is an aqueous sample.

19. The method as recited in claim 1, wherein the sample is an aqueous sample from wastewater.

20. The method as recited in claim 1, wherein the step of exposing the activated carbon to the sample comprises exposing the sample to a pathogen trap that comprises the activated carbon on a surface thereof.
